(19) United States
(12) Patent Application Publication
(10) Pub. No.: US 2025/0099199 A1
(43) Pub. Date: Mar. 27, 2025

(54) Package for a Sterile Medical Device and Assembly Method

(71) Applicant: Becton, Dickinson and Company, Franklin Lakes, NJ (US)

(72) Inventor: Gheorghe Cojocariu, Bridgewater, NJ (US)

(21) Appl. No.: 18/832,521

(22) PCT Filed: Feb. 2, 2023
(86) PCT No.: PCT/US23/61830

§ 371 Date: Jul. 24, 2024

Related U.S. Application Data
(60) Provisional application No. 63/306,563, filed on Feb. 4, 2022.

(51) Int. Cl. A61B 50/33; A61B 50/00; B65B 5/02; B65B 5/04; B65B 7/28; B65B 55/02

(52) U.S. Cl. CPC A61B 50/33; B65B 5/022; B65B 5/045; B65B 7/2871; B65B 7/2878; B65B 55/02; A61B 2050/0065

(57) ABSTRACT

A package configured to contain at least one sterile medical device includes at least one first web comprising a natural-fiber material, such as paper, paperboard, or cardboard, and at least one second web including the natural-fiber material. The at least one second web also includes at least one cavity sized to contain the at least one medical device. The at least one first web is connected to the at least one second web to provide a sterile barrier preventing microbial ingress to the at least one medical device through the package. A method of assembling a sealed sterile package containing at least one sterile medical device is also provided.

### CROSS-REFERENCE TO RELATED APPLICATION

The present application claims priority to U.S. Provisional Application Ser. No. 63/306,563, entitled “Package for a Sterile Medical Device and Assembly Method”, filed Feb. 4, 2022, the entire disclosure of which is hereby incorporate by reference in its' entirety.

### BACKGROUND OF THE INVENTION

### Field of the Invention

The present disclosure relates generally to packaging, containers, boxes, and kits that contain tools and devices used for medical procedures. More particularly, the present disclosure relates to a package or container for a sterile medical device comprising a natural-fiber material, such as paper, paperboard, or cardboard, which can be recycled in a single recycling stream.

### Description of Related Art

Medical devices are often provided in sterile barriers or packages, which can be opened by a user, such as a practitioner, clinician, or healthcare worker, at a medical treatment location. As used herein, a “healthcare worker” can be a medical professional, such as a medical technician or nurse, trained to perform a medical procedure, such as a fluid delivery or blood collection procedure. Sterile barrier packaging for medical devices usually includes components (e.g., a top web or cover, a bottom web or tray, headers, venting ports, etc.) that are made of different materials, such as plastics, paper, and/or Tyvek®. For example, some components of the packages (e.g., the bottom web in blisters and trays) can be made of composite materials, such as multilayer plastics. Other components of the packages can be made from paper, cardboard, or paperboard. The use of multiple materials in the barriers or packages means that an entire package cannot be recycled in a single recycling stream. Instead, after use, the barrier, package, or container must be deconstructed to separate different materials (e.g., paper and multiple plastics) from each other, so that the different materials can be recycled in separate, unique recycling processes or streams.

Accordingly, there is a need in the art for improved packages and containers for medical devices that can be easily sterilized to form a sealed, sterilized package and, after the medical device is removed from the package or container, can be easily recycled. Desirably, the packages or containers should also be formed from renewable and/or biodegradable materials to reduce environmental impacts of producing and discarding the packaging or containers. The packages, containers, and assembly methods of the present disclosure are configured to address these issues.

### SUMMARY OF THE INVENTION

According to an aspect of the disclosure, a package configured to contain at least one medical device includes at least one first web including a natural-fiber material and at least one second web comprising the natural-fiber material. The at least one second web includes at least one cavity sized to contain the at least one medical device. The at least one first web is connected to the at least one second web to provide a sterile barrier preventing microbial ingress to the at least one medical device through the package.

According to another aspect of the disclosure, a method of assembling a sealed sterile package containing at least one medical device includes: preparing at least one first web of the package including a natural-fiber material and at least one second web of the package including the natural-fiber material and at least one cavity sized to contain the at least one medical device for assembly. The method further includes inserting the at least one medical device into the at least one cavity of the second web and attaching an inner surface of the first web to an inner surface of the second web, thereby providing a sterile barrier preventing microbial ingress to the at least one medical device through the package.

Non-limiting illustrative examples of embodiments of the present disclosure will now be described in the following numbered clauses:
Clause 1: A package configured to contain at least one medical device, comprising: at least one first web comprising a natural-fiber material; and at least one second web comprising the natural-fiber material, wherein the at least one second web comprises at least one cavity sized to contain the at least one medical device, wherein the at least one first web is connected to the at least one second web to provide a sterile barrier preventing microbial ingress to the at least one medical device through the package.
Clause 2: The package of clause 1, wherein the package is fully recyclable in a single recycling stream.
Clause 3: The package of clause 1 or clause 2, wherein the package comprises a renewable material, such as paper, paperboard, and/or cardboard.
Clause 4: The package of any of clauses 1-3, wherein the package comprises materials that are compatible with at least one of the following sterilization processes: irradiation, gas, liquid, heat, and/or steam.
Clause 5: The package of any of clauses 1-4, wherein the at least one cavity of the at least one second web is formed by at least one of compression, thermoforming, blow molding, rotational molding, transfer molding, casting, extrusion, or injection.
Clause 6: The package of any of clauses 1-5, wherein the at least one second web has a three-dimensional shape.
Clause 7: The package of any of clauses 1-6, wherein the at least one first web is connected to the at least one second web by an adhesive.
Clause 8: The package of clause 7, wherein the at least one second web comprises a flat contour, and wherein the adhesive seals the flat contour of the at least one second web to a flat portion of an inner surface of the at least one first web.
Clause 9: The package of clause 7 or clause 8, wherein the adhesive is sealed by at least one of heat or cold sealing.
Clause 10: The package of any of clauses 7-9, wherein the adhesive is compatible with a paper recycling process.
Clause 11: The package of any of clauses 7-10, wherein the adhesive is miscible with paper and/or is water soluble to be washed away during a recycling process.
Clause 12: The package of any of clauses 1-11, wherein the at least one first web is connected to the at least one second web by an interference engagement, without adhesive.
Clause 13: The package of any of clauses 1-12, wherein an inner surface of the at least one first web and an inner surface of the at least one second web comprise porous and/or rough areas configured to be connected together to provide an interference engagement between the at least one first web and the at least one second web.
Clause 14: The package of clause 13, wherein the first web comprises a depression comprising a peripheral sloped surface configured to contact a sloped surface of the at least one cavity of the second web to form the interference engagement between the at least one first web and the at least one second web.
Clause 15: A method of assembling a sealed sterile package containing at least one medical device, the method comprising: preparing at least one first web of the package comprising a natural-fiber material and at least one second web of the package comprising the natural-fiber material, and at least one cavity sized to contain the at least one medical device for assembly; inserting the at least one medical device into the at least one cavity of the second web; and attaching an inner surface of the first web to an inner surface of the second web, thereby providing a sterile barrier preventing microbial ingress to the at least one medical device through the package.
Clause 16: The method of clause 15, wherein attaching the inner surface of the first web to the inner surface of the second web comprises applying an adhesive to portions of the inner surface of the first web and/or the second web, and drying or curing the adhesive by heat or cold sealing.
Clause 17: The method of clause 15 or clause 16, wherein attaching the inner surface of the first web to the inner surface of the second web comprises pressing rough and/or textured portions of the inner surfaces together, thereby forming an interference engagement between the at least one first web and the at least one second web.
Clause 18: The method of clause 17, wherein the interference engagement is between a sloped surface of the inner surface of the first web and a sloped surface of the at least one cavity of the second web.
Clause 19: The method of any of clauses 15-18, further comprising sterilizing the package containing the at least one medical device by at least one of the following sterilization processes: irradiation, gas, liquid, heat, and/or steam.

### BRIEF DESCRIPTION OF THE DRAWINGS

FIG. 1A is a top view of a package for a sterile medical device, according to an aspect of the present disclosure.

FIG. 1B is a perspective view of a bottom web or tray of the package of FIG. 1A, according to an aspect of the present disclosure.

FIG. 1C is a perspective view of the bottom web or tray of FIG. 1B including a medical device, according to an aspect of the present disclosure.

FIG. 2A is an exploded perspective view of another example of a package containing a sterile medical device, according to an aspect of the present disclosure.

FIG. 2B is a perspective view the package of FIG. 2A.

FIG. 2C is a perspective view of a bottom of the package of FIG. 2A.

FIG. 3A is a schematic drawing of a package showing a top web connected to a bottom web by an interference engagement, according to an aspect of the present disclosure.

FIG. 3B is an enlarged view of the package of FIG. 3A showing an interface between the top web and the bottom web.

FIG. 4 is a flow chart showing steps for assembling a sealed package enclosing a sterile medical device, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to packages 10, packaging systems, containers, boxes, and/or equipment or toolkits that provide sterile barrier packaging for tools (e.g., medical devices) used for performing medical procedures, such as fluid delivery procedures, blood draw and fluid collection procedures, and/or vascular access procedures. Medical devices that can be enclosed within the packages 10 or containers of the present disclosure can include, without limitation, surgical tools, syringes, catheters, vascular access devices, intravenous ports, and other disposable or single-use medical devices used for commonly performed medical procedures, as are known in the art.

As will be appreciated by those skilled in the art, the packages 10 of the present disclosure can provide a single easily transportable item that can be quickly obtained from a storage area and carried to a patient's bedside to perform a medical procedure. Further, the packages 10 of the present disclosure can reduce a risk of infection, improve quality of collected samples, and increase efficiency of medical procedures compared to current practices by ensuring that tools needed for performing a medical procedure are maintained in a sterile condition until ready for use.

The packages 10 of the present disclosure are made from natural-fiber materials, such as materials comprising paper, paperboard, cardboard, fiber, or pulp. In particular, the packages 10 can include webs, covers, trays, cartons, containers, and other components made from the natural-fiber materials. The different components can be connected and/or sealed together to form a sterile barrier or enclosure for the medical devices. For example, a paper-formed bottom web of the package 10 can have a flat contour that can be sealed (e.g., by heat or cold sealing) to a paper top web or cover through an adhesive layer that is compatible with paper recycling processes. Portions of the packages 10 can also be sealed together by interference engagements with or without adhesives. Once sealed, the packages 10 create a sterile barrier package for the sterile medical device.

The packages 10 and packaging systems are intended to provide a more environmentally sustainable three-dimensional enclosure for sterile medical devices compared to currently used packaging and enclosures formed from plastics. In some examples, the packages 10 comprise only one type of material (e.g., paper, paperboard, or cardboard), such as a renewable and biodegradable material, so that the entire package 10 can be recycled in a single recycling process. Further, the packages 10 can be recycled with other levels of packaging (such as shelf cartons) that are also made of paper, paperboard, or cardboard. For example, the packages 10 can be collected in the shelf cartons and recycled together, simplifying a collection process. Therefore, when combined with existing secondary (e.g., shelf cartons) and tertiary (e.g., a case carton) packaging, an entire packaging system (primary/secondary/tertiary packaging) is provided that is fully made of paper-based materials, is fully recyclable in a single recycling stream, fully renewable, and fully biodegradable.

As used herein, a “recycling stream” can refer to a single recycling process used to recycle materials of a same type. For example, natural-fiber products, such as products formed from paper, cardboard, and paperboard, may be recycled together in a single recycling stream. By contrast, products comprising glass, metal, or plastics are recycled in different recycling streams. Products containing two or more types of materials (e.g., both paper and plastics or glass) may need to be deconstructed after use so that the paper, plastics, or glass can be recycled by appropriate processes. As will be appreciated by those skilled in the art, paper (including paper, paperboard, cardboard) is one of the most commonly recycled materials in the world today, with well-developed and dedicated recycling streams available in many countries. Accordingly, infrastructure is already in place in many locations for recycling the packages 10, which are made entirely or substantially entirely from natural-fiber materials, such as paper, paperboard, and cardboard.

In some examples, the packages 10 can be made using recently developed paper forming technologies, such as technologies developed for consumer/food packaging industries. One example of a formed paper structure is the FiberForm® 3D-formable paper by BillerudKorsnäs AB of Solna, Sweden. These forming technologies can be used to build new three-dimensional primary packaging systems (e.g., blister, trays, etc.) for sterile medical devices. By contrast, use of paper in medical device packaging was previously limited to two-dimensional components (e.g., flat and/or folded structures), such as top webs or covers for blisters, trays, pouches, or bags. The recent advances in paper fiber materials and additives, as well as paper fiber forming technologies (e.g., compression, thermoforming, blow molding, rotational molding, transfer molding, casting, extrusion, and/or injection), provide for greater control and variability in three-dimensional structures that can be formed from fiber materials.

In some examples, the packages 10 and packaging systems can have versatile sterilization compatibility and can be used with many commonly-used sterilization processes, including irradiation, gas (e.g., exposure to ethylene oxide (EtO)), liquid, heat, and/or steam. Paper materials can be particularly well-suited for sterilization by irradiation, heat, and steam due to the stability of paper materials when exposed to such conditions. Paper materials are also breathable, which improves gas sterilization processes. In particular, the packages 10 can be entirely formed from breathable materials (e.g., paper), which increases efficiency of gas movement through the package 10, thereby reducing cycle times, processing costs, and environmental impacts of gas sterilization processes. Further, breathability of the packages 10 makes it easier to remove residual toxic gases, such as EtO, from the packages 10 at the end of the sterilization process and during outgassing phases.

### Packages Formed From Natural-Fiber Materials

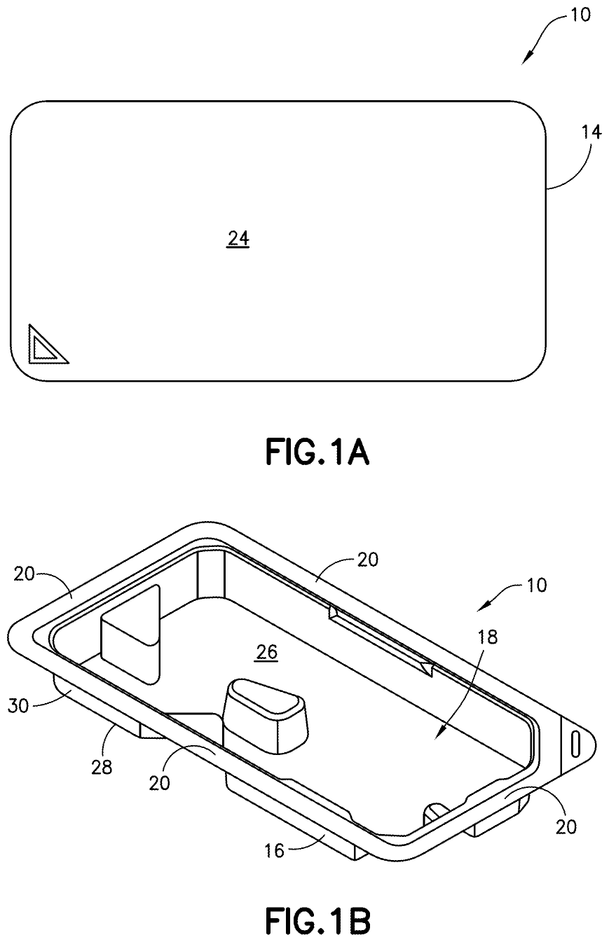

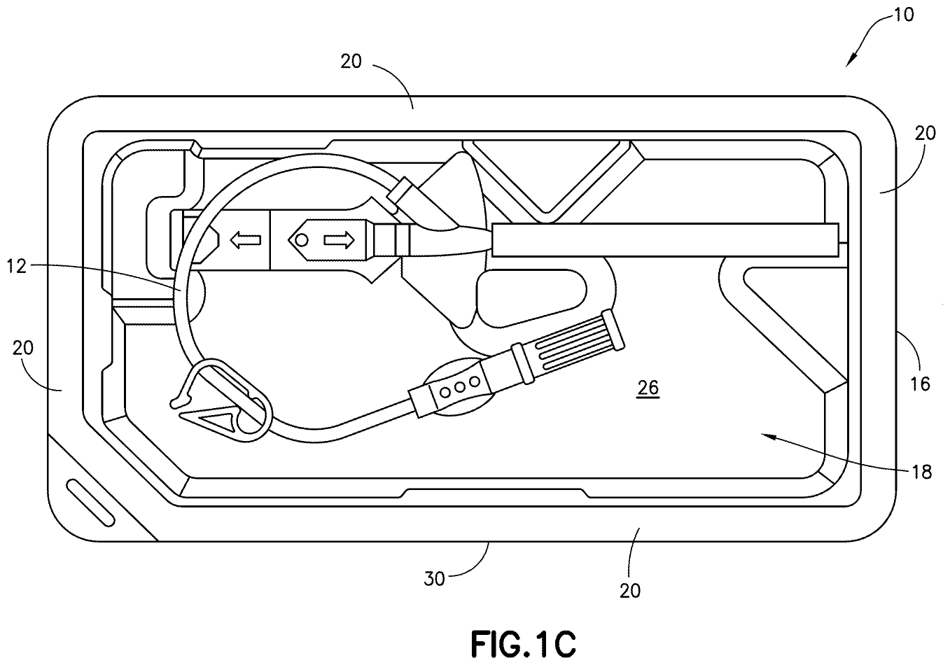

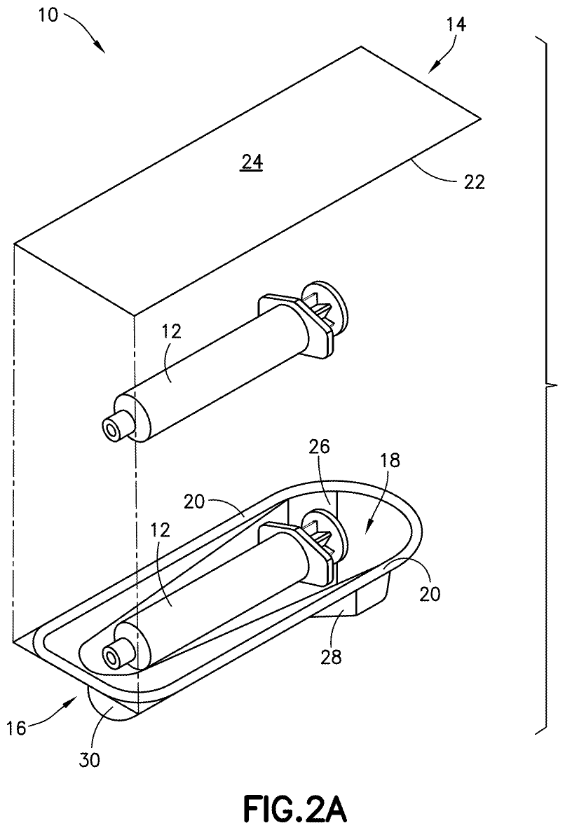

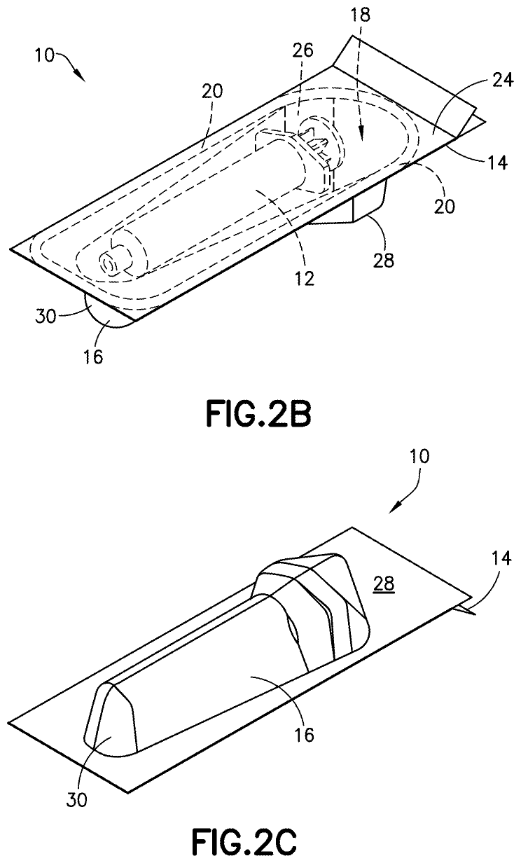

With specific reference to FIGS. 1A-2C, a package 10 or container configured to contain a medical device 12 (shown in FIGS. 1C, 2A, and 2B), such as a vascular access device or syringe, comprises a first or upper web 14, such as a cover, attached to a second or lower web 16, such as a tray. The webs 14, 16 are secured together, thereby enclosing the medical device 12 between the upper web 14 and the lower web 16. In some examples, the upper web 14 or cover can be a standard paper cover, such as a flat paper cover that is attached to a plastic tray in a conventional packaging system. In particular, as shown in FIGS. 1A-1C, the upper web 14 can comprise a flat or substantially flat sheet including an outer surface 24, an inner surface 22, and a peripheral edge extending between the surfaces 22, 24. The lower web 16 can be a three-dimensional structure including an inner surface 26, configured contact the inner surface 22 of the upper web 14 to securely hold the medical device 12 within the package 10, an outer surface 28, and side surfaces 30 between the inner surface 26 and the outer surface 28.

As used herein, securely containing or holding the medical device(s) 12 within the package 10 means that the webs 14, 16 are held together tightly enough that the package 10 can be jostled, shaken, inverted, rotated, or otherwise moved without the medical device 12 falling out of the package 10. The medical device 12 can be removed from the packaging 10 by, for example, pulling the upper web 14 away from the lower web 16, ripping or otherwise deforming the package 10, or similar actions. Once the upper web 14 is removed or separated from the lower web 16, the package 10 is in an open or ready-for-use position, where the healthcare worker can access the medical device(s) 12 contained in the package 10, and can begin performing a medical procedure.

As used herein, a “web” refers to a two-dimensional or a three-dimensional structure formed by bending, folding, or otherwise deforming portions of a flat sheet to form a structure having a desired shape and curvature. As previously described, the webs 14, 16 are formed from natural-fiber materials, such as paper, paperboard, or cardboard. In other examples, the webs 14, 16 can be formed from other reproducible and/or recyclable materials formed from reusable and/or repurposed fibers, as are known in the art. In some examples, the webs 14, 16 can be formed from foldable or moldable paper materials, such as the FiberForm® formable paper by BillerudKorsnäs AB.

As shown in FIGS. 1A-1C, the upper web 14 is a flat cover or cover sheet configured to be adhered over the lower web 16. The lower web 16 can include one or more cavities 18, depressions, or chambers sized to contain the medical devices 12. The cavities 18 of the lower web 16 can be formed by any suitable molding, forming, bending, or deforming process including, without limitation, compression, thermoforming, blow molding, rotational molding, transfer molding, casting, extrusion, or injection. In some examples, the lower web 16 can be formed on an existing production line (such as existing thermoforming line). Alternatively, the lower web 16 can be formed on a dedicated production line using fiber molding from liquid (slurry) or dry form. The produced lower webs 16 can be sealed to the top webs 14 using existing sealing equipment, such as equipment used for drying or curing an adhesive by heat or cold sealing. Alternatively, as described herein, a closure between the webs 14, 16 can be formed by interference fit or engagement between porous or rough surfaces of the webs 14, 16.

In some examples, the cavities 18 of the lower web 16 are elongated cavities sized to receive elongated medical devices, such as a syringe, syringe barrel, or fluid collection tube. The lower web 16 can also include cavities 18 of different shapes or configurations. For example, the lower web 16 can include smaller cavities 18 sized to receive medical accessories, such as cotton swabs, wipes, or other cleaning or disinfecting tools used during medical procedures. In some examples, some of the cavities 18 of the lower web 16 can also include holding or support structures, such as tube stands or tube holders, sized to receive a sample collection tubes and to support the sample collection tubes in an upright position relative to the lower web 16 during a medical procedure.

The upper web 14 is connected to the lower web 16 in order to enclose the medical device 12 within the cavity 18 of the lower web 16. When the webs 14, 16 are connected, the webs 14, 16 provide the sterile barrier, which can prevent microbial ingress to the medical device 12 contained within the package 10. In some examples, the upper web 14 can be sealed and/or attached to the lower web 16 by mechanical fasteners or adhesives. For example, the upper web 14 can be connected to the lower web 16 by an adhesive layer positioned between the upper web 14 and the lower web 16. In some examples, the adhesive or adhesive layer can be miscible with paper and/or water soluble to be washed away during a recycling process. Also, the amount of adhesive used for securing the upper web 14 to the lower web 16 can be controlled, so that the entire package 10, including the adhesive, can be recycled by a paper recycling process. For example, the adhesive can be limited to from about 5% to about 10% of the entire package 10, so that the adhesive does not contaminate the recycling process.

In some examples, in order to secure the upper web 14 to the lower web 16 by the adhesive, the lower web 16 can include a flat contour 20. As shown in FIGS. 1A-2C, the flat contour 20 extends around a periphery of the lower web 16. In other examples, the flat contour 20 may extend around or between cavities 18 of the lower web 16. The flat contour 20 can be configured to contact flat portions of the inner surface 22 of the upper web 14. For example, the adhesive can be applied to the flat contour 20 and/or to portions of the inner surface 22 of the upper web 14. The contour 20 and the inner surface 22 can then be pressed together to secure the upper web 14 to the lower web 16. In some examples, heat or cold (e.g., heat sealing or cold sealing) can be applied to the webs 14, 16 and/or adhesive to cure or seal the adhesive. Once the adhesive is dry or cured, the package 10 provides the sterile barrier for the medical device 12 until ready for use.

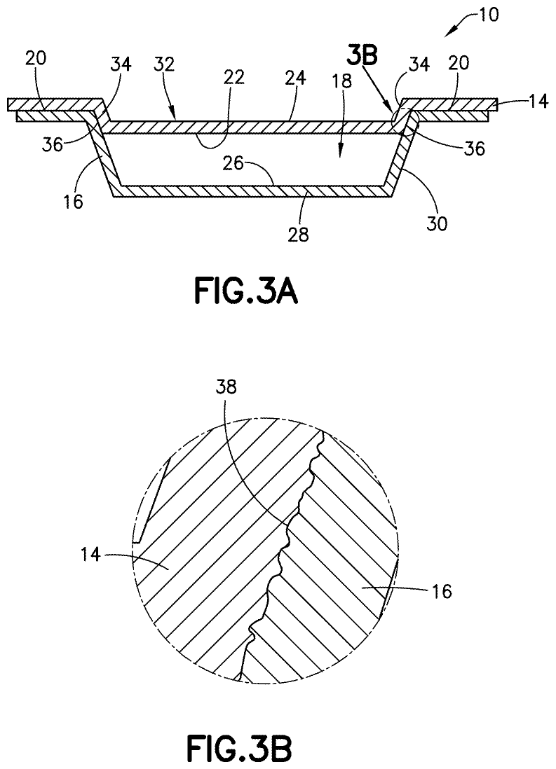

With reference to FIGS. 3A and 3B, in other examples, the upper web 14 can be connected to the lower web 16 by an interference fit or an interference engagement with or without adhesive. For example, the inner surface 22 of the upper web 14 and the inner surface 26 of the lower web 16 can comprise porous and/or rough portions or areas that contact each other to form the interference fit or engagement between the upper web 14 and the lower web 16. As used herein, a porous and/or rough surface refers to an irregular surface having texturing, pores, or spaces, such that gaps are present between the inner surface 22 of the upper web 14 and the inner surface 26 of the lower web 16, when the inner surfaces 22, 26 are pressed together. In particular, pore size and distribution of the inner surfaces 22, 26 can be selected so that the upper web 14 attaches to the lower web 16 in a secure manner. Due to the irregularity of the porous and/or rough portions or areas, there is a tortuous contact area or tortuous path 38 (shown in FIG. 3B) between the webs 14, 16, which creates friction between the webs 14, 16, thereby securing the webs 14, 16 together. Also, the created contact area or tortuous path 38 can be configured to allow for gas sterilization, permitting gas to pass through gaps in the contact area or path 38, while preventing microbial ingress into the package 10 through the gaps due to the shape, size, and/or configuration of the gaps of the tortuous path 38. Desirably, the webs 14, 16 are also sufficiently rigid to maintain the interference fit or engagement between the inner surfaces 22, 26 of the webs 14, 16 for extended periods of time, until the medical device 12 is ready for use.

More specifically, in some examples, as shown in FIGS. 3A and 3B, the upper web 14 comprises a molded depression 32 comprising a peripheral sloped surface 34 configured to contact a sloped surface 36 of the cavity 18 of the lower web 16. The sloped surfaces 34, 36 can include the previously described porous and/or rough portions. Therefore, the contact between the sloped surfaces 34, 36 provides the interference fit or engagement between the upper web 14 and the lower web 16. As a result of the interference fit or engagement, the medical device 12 can be sealed within the cavity 18 of the lower web 16 forming the sterile barrier without using adhesives. As in previous examples, when the webs 14, 16 are connected together, the package 10 prevents microbial ingress through the package 10 to maintain the medical device 12 in a sterile condition.

### Method of Assembly for a Package or Container

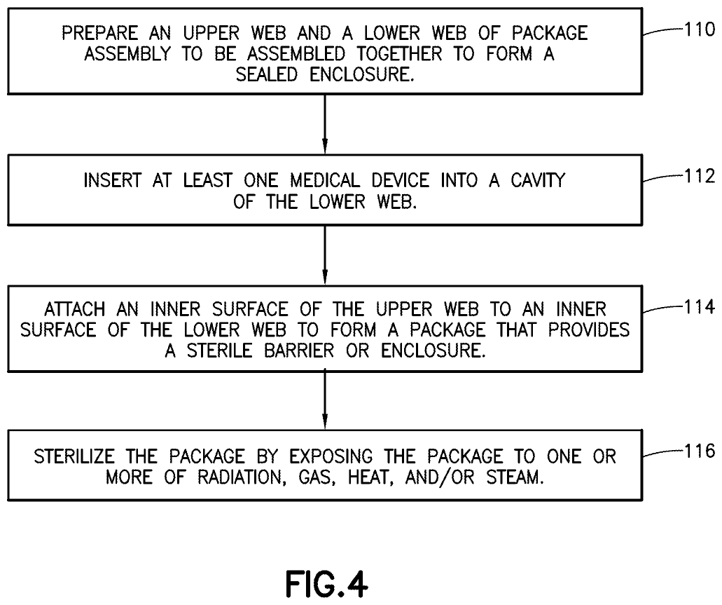

FIG. 4 is a flow chart showing steps for assembling the sterile package 10 containing the medical device 12. As shown in FIG. 4, at step 110, a first or upper web 14 and a second or lower web 16 are prepared for assembly. For example, flat sheets of a natural-fiber material, such as paper, paperboard, or cardboard, can be cut to a desired size and shape for the webs 14, 16. The sheets can also be molded, pressed, or otherwise deformed to a final shape for the webs 14, 16. For example, the cavity 18 can be molded into a flat sheet to form the lower web 16. Also, the depression 32 that corresponds in size and shape to the cavity 18 can be molded to a flat sheet to form the upper web 14. Once the webs 14, 16 are prepared, at step 112, medical devices 12 are inserted into the cavities 18 of the lower web 16. At step 114, once the medical devices 12 are in place, the inner surface 22 of the upper web 14 is attached to the inner surface 26 of the lower web 16, which provides the sterile barrier preventing microbial ingress to the medical device 12 through the package 10. For example, as previously described, the upper web 14 can be connected to the lower web 16 by an adhesive. Alternatively or in addition, the upper web 14 and the lower web 16 can be connected by an interference fit or interference engagement between rough and/or porous portions or areas of the inner surface 22 of the upper web 14 and the inner surface 26 of the lower web 14.

At step 116, after the webs 14, 16 are attached together, the formed and sealed package 10 can be sterilized. For example, sterilizing the package 10 can include exposing the package 10 to one or more of the following sterilization processes: irradiation, gas (exposure to EtO, etc.), heat, liquid, and/or steam. As previously discussed, packages 10 formed from natural-fiber materials, such as paper, paperboard, or cardboard, are well-suited for use with irradiation, heat, and steam because paper materials are generally stable when exposed to such conditions. Packages 10 formed from paper materials are also well-suited for gas sterilization because paper structures are breathable, which allows the gas to enter and escape from the sealed package 10, thereby improving efficiency of the gas sterilization process.

While examples of the packages, packaging systems, containers, and methods are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A package configured to contain at least one medical device, comprising:
at least one first web comprising a natural-fiber material; and
at least one second web comprising the natural-fiber material, wherein the at least one second web comprises at least one cavity sized to contain the at least one medical device,
wherein the at least one first web is connected to the at least one second web to provide a sterile barrier preventing microbial ingress to the at least one medical device through the package.

2. The package of claim 1, wherein the package is fully recyclable in a single recycling stream.

3. The package of claim 1, wherein the package comprises a renewable material, such as paper, paperboard, and/or cardboard.

4. The package of claim 1, wherein the package comprises materials that are compatible with at least one of the following sterilization processes: irradiation, gas, liquid, heat, and/or steam.

5. The package of claim 1, wherein the at least one cavity of the at least one second web is formed by at least one of compression, thermoforming, blow molding, rotational molding, transfer molding, casting, extrusion, or injection.

6. The package of claim 1, wherein the at least one second web has a three-dimensional shape.

7. The package of claim 1, wherein the at least one first web is connected to the at least one second web by an adhesive.

8. The package of claim 7, wherein the at least one second web comprises a flat contour, and wherein the adhesive seals the flat contour of the at least one second web to a flat portion of an inner surface of the at least one first web.

9. The package of claim 7, wherein the adhesive is sealed by at least one of heat or cold sealing.

10. The package of claim 7, wherein the adhesive is compatible with a paper recycling process.

11. The package of claim 7, wherein the adhesive is miscible with paper and/or is water soluble to be washed away during a recycling process.

12. The package of claim 1, wherein the at least one first web is connected to the at least one second web by an interference engagement, without adhesive.

13. The package of claim 1, wherein an inner surface of the at least one first web and an inner surface of the at least one second web comprise porous and/or rough areas configured to be connected together to provide an interference engagement between the at least one first web and the at least one second web.

14. The package of claim 13, wherein the first web comprises a depression comprising a peripheral sloped surface configured to contact a sloped surface of the at least one cavity of the second web to form the interference engagement between the at least one first web and the at least one second web.

15. A method of assembling a sealed sterile package containing at least one medical device, the method comprising:
preparing at least one first web of the package comprising a natural-fiber material and at least one second web of the package comprising the natural-fiber material and at least one cavity sized to contain the at least one medical device for assembly;
inserting the at least one medical device into the at least one cavity of the second web; and
attaching an inner surface of the first web to an inner surface of the second web, thereby providing a sterile barrier preventing microbial ingress to the at least one medical device through the package.

16. The method of claim 15, wherein attaching the inner surface of the first web to the inner surface of the second web comprises applying an adhesive to portions of the inner surface of the first web and/or the second web, and drying or curing the adhesive by heat or cold sealing.

17. The method of claim 15, wherein attaching the inner surface of the first web to the inner surface of the second web comprises pressing rough and/or textured portions of the inner surfaces together, thereby forming an interference engagement between the at least one first web and the at least one second web.

18. The method of claim 17, wherein the interference engagement is between a sloped surface of the inner surface of the first web and a sloped surface of the at least one cavity of the second web.

19. The method of claim 15, further comprising sterilizing the package containing the at least one medical device by at least one of the following sterilization processes: irradiation, gas, liquid, heat, and/or steam.
